(19) United States
(12) Patent Application Publication
(10) Pub. No.: US 2022/0175270 A1
(43) Pub. Date: Jun. 9, 2022

(54) BREATH MONITORING APPARATUS FOR PRODUCING A CAPNOGRAM

(71) Applicant: Keith Simpson, Abbotskerswell (GB)

(72) Inventor: Keith Simpson, Abbotskerswell (GB)

(21) Appl. No.: 17/682,176

(22) Filed: Feb. 28, 2022

Related U.S. Application Data
(63) Continuation of application No. PCT/GB2020/052118, filed on Sep. 4, 2020.

(30) Foreign Application Priority Data
Sep. 5, 2019 (GB) 1912772.9

(51) Int. Cl. A61B 5/087; A61B 5/08; A61B 5/00; A61M 16/06; A61M 16/01; A61M 16/00

(52) U.S. Cl. CPC A61B 5/087; A61B 5/082; A61B 5/743; A61M 16/06; A61M 16/01; A61M 2205/584; A61B 2562/0247; A61B 2562/0271; A61B 2562/0204; A61M 2230/432; A61M 2207/00; A61M 16/0003

(57) ABSTRACT

This apparatus produces a capnogram for a patient, comprising airflow means arrangeable such that air flowing into and out of a patient during a breathing cycle of the patient are conducted through the airflow means; a CO2 monitor (50) for repeatedly determining the presence, and level, of CO2 in the air within the airflow means; an airflow direction sensor for distinguishing between periods when air is flowing into the patient, when air is flowing out of the patient, and when air in the airflow means is stationary; a processor for producing a graph on a display of time versus an indication of CO2 levels, wherein the display includes different graphical qualities to distinguish between the level of CO2 in the air flowing out of the patient, the level of CO2 in the stationary air between breaths, and the level of CO2 in the air flowing into the patient.

### RELATED APPLICATIONS

This application claims the benefit under 35 U.S.C. § 120, and is a continuation, of co-pending International Application PCT/GB2020/052118, filed Sep. 4, 2020 and designating the US, which claims priority to GB Applications 1912772.9, filed Sep. 5, 2019, such GB Application also being claimed priority to under 35 U.S.C. § 119. These GB and International applications are incorporated by reference herein in their entireties.

### FIELD

The present invention relates generally to a breath monitoring apparatus for producing a capnogram and a method of producing a capnogram for a patient, and finds particular, although not exclusive, utility in the field of capnography for humans and animals during periods when they may be anaesthetised.

### BACKGROUND

Capnography includes devices for monitoring CO2 levels in breath. The levels are typically shown in graph form. The graphs are produced in real-time (or with very slight delays of the order of a few seconds). These provide information to the anaesthetist or other clinician enabling them to provide better care to the patient and to prevent potential damage to the patient through inhaling CO2 rich air.

Known devices are able to produce capnograms showing CO2 levels over time, however there is no information as to which direction the air is moving, or if it is stationary. Accordingly, the patient may be breathing in CO2 rich air which they have already breathed-out, or may have episodes of no breathing, neither of which can be determined from the capnogram.

### SUMMARY

In a first aspect, the present invention provides a breath monitoring apparatus for producing a capnogram for a patient, comprising airflow means arrangeable such that air flowing into and out of a patient during a breathing cycle of the patient are conducted through the airflow means; a CO2 monitor for repeatedly determining the presence, and level, of CO2 in the air within the airflow means; an airflow direction sensor located within the airflow means, the airflow direction sensor for providing data distinguishing between periods when air is flowing into the patient, when air is flowing out of the patient, and when air in the airflow means is stationary; a processor for producing a graph on a display of an indication of CO2 levels versus time, wherein the display includes different graphical qualities to distinguish between the level of CO2 in the air flowing out of the patient, the level of CO2 in the stationary air between breaths, and the level of CO2 in the air flowing into the patient.

In this manner, the clinician may visually be able to check the level of CO2 associated with the patient's breathing in, breathing out and any pauses in breathing. The clinician may visually be able to check the degree of CO2 rebreathing by the patient. The display provides information in an easily, and immediately, understandable format allowing for rapid response to potentially life-threatening situations.

The airflow means may include a tube or, a mask and a tube. The tube may be arranged to be insertable directly into the airway of the patient, such as an endotracheal tube. Alternatively, a mask may be used to cover the mouth and nostrils of the patient and a tube may lead from it, directing the breath.

The airflow direction sensor may include one or more of a pressure sensor, a temperature sensor, a micro-flow sensor, and an acoustic sensor. If a pressure sensor is employed it may detect airflow direction by means of a differential pressure sensor attached to two apertures provided in the airflow means tube arranged linearly with respect to one another such that one aperture is downstream of the other. The apertures may be connected to the sensor via tubes. Other means of determining airflow direction are contemplated.

The processor may be arranged to determine the airflow direction from signals sent by the airflow direction sensor. For example, if a differential pressure sensor is used, a higher pressure in one aperture compared to the other aperture may indicate breathing out and vice-versa may indicate breathing in. A balanced pressure across the two apertures may indicate no, or very little, breathing occurring.

The processor may be configured to display the level of CO2 as received from the CO2 monitor at a first sampling time, with the airflow direction data received from the airflow direction sensor from a second sampling time. The first sampling time may be the same as the second sampling time. In this regard the two may be only a few milliseconds apart yet still considered to be simultaneous. This may occur in a mainstream configuration where the CO2 monitor and airflow direction sensor are both located within the same airflow means and located relatively close to one another. However, in a sidestream configuration, where the CO2 monitor is located in a tube connected to, but not located directly in, the airflow means, the CO2 level may relate to an airflow direction of an earlier period. In other words, there may be a delay between the two readings. In this regard, the processor may be configured to display the level of CO2, as received from the CO2 monitor a few seconds later than the airflow direction data.

This may allow for the delay in the air reaching the CO2 monitor to ensure that the two sets of data relate to the same air at the same time.

The delay between the two readings may be 10 milliseconds to 30 seconds.

The level of CO2 may be expressed on the display as either or both of a percentage of CO2 in the airflow, and a partial pressure of CO2.

The display may include different colours to distinguish between the level of CO2 in the air flowing out of the patient, the level of CO2 in the stationary air between breaths, and the level of CO2 in the air flowing into the patient. For instance, a white area under the graph line may be used to indicate breathing out, yellow may indicate no airflow, and blue may indicate breathing in. Other colours and combinations of colours are contemplated.

The airflow direction sensor and the CO2 monitor may be combined in a single unit.

In a second aspect, the invention provides a method of producing a capnogram for a patient, comprising the steps of:

(a) providing a breath monitoring apparatus according to the first aspect;

(b) arranging the airflow means to conduct the airflow into and out of a patient during a breathing cycle;

(c) measuring the direction of airflow during the patient's breathing to distinguish between breathing in, breathing out, and no airflow;

(d) determining the presence, and level, of CO2 in the air flowing in and out of the patient during the breathing cycle;

(e) producing a graph on a display showing an indication of CO2 levels versus time;

(f) providing different graphical qualities to the graph to distinguish between the CO2 as measured in the air flowing out of the patient, the CO2 as measured in the stationary air between breaths, and the CO2 as measured in the air flowing into the patient.

In this manner, the clinician may visually be able to check the level of CO2 associated with the patient's breathing in, breathing out and any pauses in breathing. The clinician may visually be able to check the degree of CO2 rebreathing by the patient. The display provides information in an easily, and immediately, understandable format allowing for rapid response to potentially life-threatening situations.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a schematic diagram showing apparatus for producing capnograms; and

FIG. 2 is an example capnogram.

### DETAILED DESCRIPTION

The present invention will be described with respect to certain drawings but the invention is not limited thereto but only by the claims. The drawings described are only schematic and are non-limiting. Each drawing may not include all of the features of the invention and therefore should not necessarily be considered to be an embodiment of the invention. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein. Likewise, method steps described or claimed in a particular sequence may be understood to operate in a different sequence.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Similarly, it is to be noticed that the term “connected”, used in the description, should not be interpreted as being restricted to direct connections only. Thus, the scope of the expression “a device A connected to a device B” should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other. For instance, wireless connectivity is contemplated.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

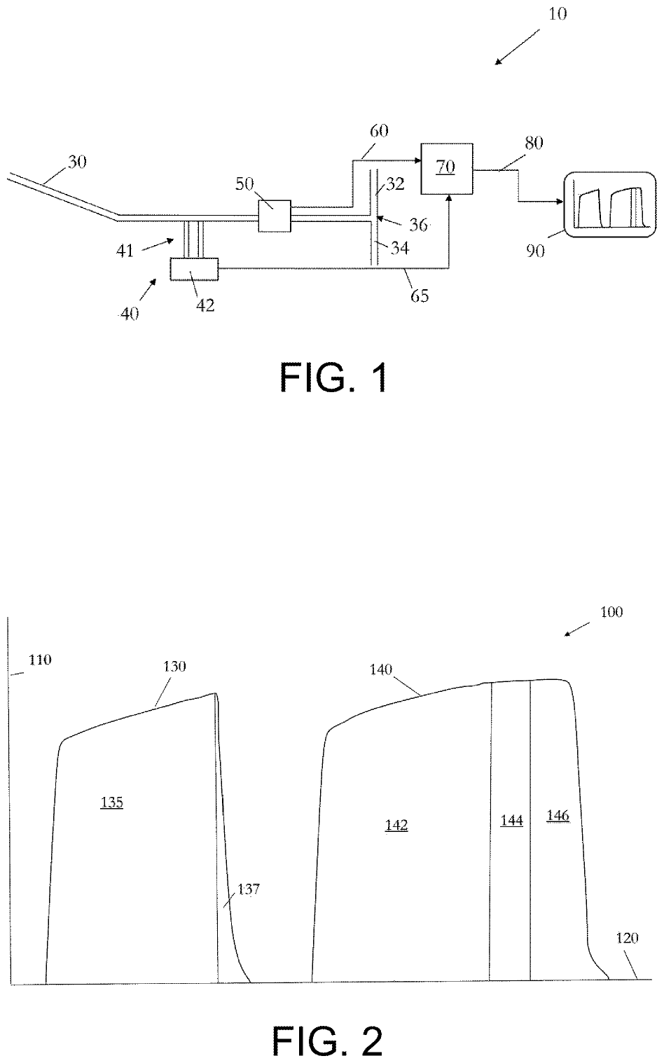

In FIG. 1, a schematic diagram of apparatus 10 for producing a capnogram is shown. An endotracheal tube 30 is provided for placing within a patient's trachea. It leads to a T junction 36 where it diverges into two tubes 32, 34. One tube 32 allows air from an anaesthetic machine (not shown) to enter the tube 30. The other tube 34 allows breathed out air to be exhausted from the system. Valves may be arranged in the various tubes 30, 32, 34, to control the flow of air therethrough.

Air within the tube 30 may flow in either direction.

It is to be understood that instead of an endotracheal tube a mask (not shown) may be provided for placing over the patient's mouth and nose.

A CO2 monitor 50, for repeatedly determining the level of CO2 in the air within the tube 30, is arranged in that tube. The CO2 levels are directed to a processor 70 via connection means 60.

The apparatus is shown in a “mainstream configuration” whereby the CO2 monitor 50 is arranged in the tube 30 leading from the mask. It will be understood that in an alternative “sidestream configuration” (not shown) the CO2 monitor 50 may be arranged in a tube leading off from the side of the tube 30 leading from the mask.

The airflow direction sensor 40 comprises two tubes 41, which may be fine-bore, leading off the endotracheal tube 30. These tubes 41 contain the same fluid (air) as the endotracheal tube 30. These tubes lead to a device 42 for determining the pressure difference in the fluid (air) within those two tubes. The result of that pressure difference is communicated to the processor 70 via connection means 65.

Although a mask 20 is shown it is to be understood that an endotracheal tube may be used in its place.

The processor 70 is arranged to determine the airflow direction within the tube 30 from the pressure difference communicated to it by the device 42. The processor then creates data signals for producing a capnogram based on the information received from the pressure sensor and CO2 monitor. The capnogram is displayed on a VDU screen 90 which is connected to the processor 70 via connection means 80.

An example capnogram 100 is shown in FIG. 2. The y-axis is percentage CO2 in the monitored air. The x-axis is time

A first graph 130 of CO2 is shown on the left. The CO2 levels rise relatively quickly, stay relatively constant for a time period and then reduce relatively quickly. On the VDU the area 135 under the graph will appear white in colour to indicate that the airflow direction is outwardly from the patient and that therefore the patient has breathed out air including this level of CO2. However, the last portion 137 from the peak of CO2 to when the CO2 level is at a minimum will be coloured blue to indicate that the patient is breathing in. As can be seen, this portion is minimal and may be related to “re-breathing” expired CO2 which is present in the bi-directional tube 30. The remainder of the time in which the patient is breathing in will be indicated by a blue line at zero percentage (not visible in the Figure) because there is no CO2 present.

A second graph 140 is shown on the right. Again, the CO2 levels rise relatively quickly, stay relatively constant for a time period and then reduce relatively quickly. In a similar manner, the area 142 under the graph will appear white in colour to indicate that the airflow direction is outwardly from the patient and that therefore the patient has breathed out air including this level of CO2. However, this white area only extends for approximately 60% of the total area under the graph 140.

An area 144 is shown immediately to the right of the white area 142. This will be coloured yellow on the VDU. This indicates that there is no movement of air. It is created by the patient's pause between breathing out and breathing in. The CO2 level remains relatively constant as is shown by the relatively horizontal nature of the line of the graph 140 at the top of this yellow area 144. This yellow area 144 extends for approximately 20% of the total area under the graph 140.

Another area 146 is shown immediately to the right of the yellow area 144. This will be coloured blue on the VDU. This indicates that the direction of airflow is inwardly towards the patient and that therefore the patient has breathed in air including this level of CO2. This blue area 146 extends for approximately 20% of the total area under the graph 140. The level of CO2 falls relatively quickly as the patient continues breathing in with no CO2 present.

Without the ability to determine the direction of airflow a clinician would only see the graph on the right as being of one colour and would not be able to determine if the CO2 levels shown relate to breathing in, breathing out or the natural pause between the two.

By being able to determine the airflow direction and indicate it on the capnogram a clinician may be able to take steps to prevent the patient from breathing in air containing levels of CO2 which may be harmful to the patient.

1. A breath monitoring apparatus for producing a capnogram for a patient, comprising a tube arrangeable such that air flowing into and out of a patient during a breathing cycle of the patient are conducted through the tube; a CO2 monitor for repeatedly determining the presence, and level, of CO2 in the air within the tube; an airflow direction sensor located within the tube, the airflow direction sensor for providing data distinguishing between periods when air is flowing into the patient, when air is flowing out of the patient, and when air in the tube is stationary; a processor for producing a graph on a display of an indication of CO2 levels versus time, wherein the display includes different graphical qualities to distinguish between the level of CO2 in the air flowing out of the patient, the level of CO2 in the stationary air between breaths, and the level of CO2 in the air flowing into the patient.

2. The breath monitoring apparatus of claim 1, wherein the tube includes a mask.

3. The breath monitoring apparatus of claim 1, wherein the airflow direction sensor includes one or more of a pressure sensor, a temperature sensor, a micro-flow sensor, and an acoustic sensor.

4. The breath monitoring apparatus of claim 1, wherein the processor is arranged to determine the airflow direction from signals sent by the airflow direction sensor.

5. The breath monitoring apparatus of claim 1, wherein the processor is configured to display the level of CO2, as received from the CO2 monitor at a first sampling time, with the airflow direction data received from the airflow direction sensor from a second sampling time.

6. The breath monitoring apparatus of claim 5, wherein the first sampling time is the same as the second sampling time.

7. The breath monitoring apparatus of claim 5, wherein the first sampling time is later than the second sampling time.

8. The breath monitoring apparatus of claim 7, wherein the first sampling time is later than the second sampling time by 10 milliseconds to 30 seconds.

9. The breath monitoring apparatus of claim 1, wherein the level of CO2 is expressed on the display as either or both of a percentage of CO2 in the airflow, and a partial pressure of CO2.

10. The breath monitoring apparatus of claim 1, wherein the display includes different colours to distinguish between the level of CO2 in the air flowing out of the patient, the level of CO2 in the stationary air between breaths, and the level of CO2 in the air flowing into the patient.

11. The breath monitoring apparatus of claim 1, wherein the airflow direction sensor and the CO2 monitor are combined in a single unit.

12. A method of producing a capnogram for a patient, comprising the steps of:
(a) providing a breath monitoring apparatus according to claim 1;
(b) arranging the tube to conduct the airflow into and out of a patient during a breathing cycle;
(c) measuring the direction of airflow during the patient's breathing to distinguish between breathing in, breathing out, and no airflow;
(d) determining the presence, and level, of CO2 in the air flowing in and out of the patient during the breathing cycle;
(e) producing a graph on a display showing an indication of CO2 levels versus time;
(f) providing different graphical qualities to the graph to distinguish between the CO2 as measured in the air flowing out of the patient, the CO2 as measured in the stationary air between breaths, and the CO2 as measured in the air flowing into the patient.

13. A breath monitoring apparatus for producing a capnogram for a patient, comprising a tube arrangeable such that air flowing into and out of a patient during a breathing cycle of the patient are conducted through the tube; a CO2 monitor for repeatedly determining the presence, and level, of CO2 in the air within the tube; an airflow direction sensor located within the tube, the airflow direction sensor for providing data distinguishing between periods when air is flowing into the patient, when air is flowing out of the patient, and when air in the tube is stationary; a processor for producing a graph on a display of an indication of CO2 levels versus time, wherein the display includes different colours to distinguish between the air flowing out of the patient, the stationary air between breaths, and the air flowing into the patient.

14. The breath monitoring apparatus of claim 13, wherein the tube includes a mask.

15. The breath monitoring apparatus of claim 13, wherein the airflow direction sensor includes one or more of a pressure sensor, a temperature sensor, a micro-flow sensor, and an acoustic sensor.

16. The breath monitoring apparatus of claim 13, wherein the processor is arranged to determine the airflow direction from signals sent by the airflow direction sensor.

17. The breath monitoring apparatus of claim 13, wherein the processor is configured to display the level of CO2, as received from the CO2 monitor at a first sampling time, with the airflow direction data received from the airflow direction sensor from a second sampling time.

18. The breath monitoring apparatus of claim 17, wherein the first sampling time is the same as the second sampling time.

19. The breath monitoring apparatus of claim 17, wherein the first sampling time is later than the second sampling time.

20. The breath monitoring apparatus of claim 19, wherein the first sampling time is later than the second sampling time by 10 milliseconds to 30 seconds.

21. The breath monitoring apparatus of claim 13, wherein the level of CO2 is expressed on the display as either or both of a percentage of CO2 in the airflow, and a partial pressure of CO2.

22. The breath monitoring apparatus of claim 13, wherein the airflow direction sensor and the CO2 monitor are combined in a single unit.

23. The breath monitoring apparatus of claim 13, wherein the different colours included by the display are:
the colour white to indicate the air flowing out of the patient;
the colour yellow to indicate the stationary air between breaths; and
the colour blue to indicate the air flowing into the patient.
